(19) United States
(12) Patent Application Publication
(10) Pub. No.: US 2011/0212119 A1
(43) Pub. Date: Sep. 1, 2011

(54) METHODS FOR MAKING HIV VACCINES AND RELATED COMPOSITIONS

(75) Inventor: Richard B. Williams, South Pasadena, CA (US)

(73) Assignee: PROTEONOVA, INC., Pasadena, CA (US)

(21) Appl. No.: 13/126,935

(22) PCT Filed: Oct. 22, 2009
(86) PCT No.: PCT/US2009/061720

§ 371 Date: Apr. 29, 2011

Related U.S. Application Data
(60) Provisional application No. 61/110,450, filed on Oct. 31, 2008.

(51) Int. Cl. A61K 39/385; C40B 30/04; A61P 31/18

(52) U.S. Cl. 424/188.1; 506/9

(57) ABSTRACT

Provided herein are methods and kits for making a targeted therapeutic for prevent and treating HIV infection. In one of these methods, the method includes preparing a library of soluble peptides, and screening the library for proteins that bind to the region of CD4 that binds gp120. In this particular method, these proteins are used as the antigen to make the HIV vaccine.

### BACKGROUND OF THE INVENTION

1. Field of the Invention

The present invention relates to methods for making therapeutics tailored to human immunodeficiency virus (HIV), as well as methods of using such therapeutics to prevent and treat HIV infection and Acquired Immune Deficiency Syndrome (AIDS).

2. Description of the Related Art

AIDS has been described as a modern plague. The cumulated estimated number of deaths of persons with AIDS in the United States and dependent areas is more than 500,000. A study by the United States Centers for Disease Control and Prevention (CDC) reveals that there were more than a million people—an estimated 1,106,400 adults and adolescents—living with HIV infection in the United States at the end of 2006. However, the true impact of the disease has yet to be felt. The virus may remain latent in infected individuals for five or more years before symptoms appear. Many Americans may unknowingly be infected and capable of infecting others who might come into contact with their body fluids. Virtually every person who contracts the virus will develop AIDS and may eventually die as a consequence. Thus, if unchecked, the personal, social and economic impact of AIDS will be enormous.

The causative agent of AIDS is Human Immunodeficiency Virus Type 1 (HIV-1, or HIV). The intact HIV virion is roughly spherical and is approximately 110 nm in diameter. The virion has an outer membrane covered with knobs or spikes made up of glycoprotein, gp160/120. In addition, there exists a transmembrane protein termed gp41. Inside the virion are two structural proteins: an outer shell composed of the phosphoprotein p17 and an inner nucleoid or central core made up of the phosphoprotein, p24. The viral RNA is present inside the core along with two copies of the reverse transcriptase enzyme, RT or p65, which is necessary for the synthesis of viral DNA from the RNA template. In an infected person, antibodies are made to each of the aforementioned protein components and exist in characteristic concentrations throughout the course of the disease.

Most of the current HIV vaccines have attempted to elicit a humoral or cell mediated immune response to epitopes expressed on the HIV envelope such as gp 41, and domains on the gp120: CI, C5, V1/V2, V3, 2G12, b12 (PNAS, USA 94(19):10018-10023, 1997). To date however, none of the vaccine attempts created to these epitopes have proved effective at preventing HIV infection in humans under physiological conditions.

The problems that have to be overcome to make an effective vaccine against HIV are formidable. For example, the virus has very high sequence diversity. It has also evolved tricky immune avoidance defenses, such as an ability to mask neutralizing epitopes and it down regulates MHC which is required for antigen presentation. Further, HIV's high mutation rate enables it to mutate to forms that escape the immune system, and most clever and insidious of all, it targets, cripples, and kills the T helper cells, the very cells that initiate the acquired immune response to foreign antigens (Science, 320:760-764, 2008). As a result, it has been very difficult to make effective HIV vaccines.

### SUMMARY OF THE INVENTION

In various aspects, methods are provided herein for developing therapeutics useful in preventing and treating HIV infection by targeting the region of gp120 that binds CD4. Also provided are methods for preventing and treating HIV infection by administering a vaccine or antibody produced by methods described herein.

Various embodiments of a method of the present teachings comprise: preparing a population of soluble proteins; selecting a subpopulation of proteins from said population, wherein said subpopulation of proteins comprises proteins that bind CD4; and selecting at least one protein from said subpopulation, wherein said at least one protein does not bind to CD4 when CD4 is bound to gp120. In some embodiments, the population of soluble proteins comprises proteins linked to their cognate mRNA. In some embodiments, the proteins of the library are each about 100 amino acids in length.

In some embodiments, the step of selecting a subpopulation of proteins from said population comprises: screening said population of soluble proteins against a CD4 protein attached to a solid substrate; and selecting proteins binding to said CD4 protein.

In some embodiments, the step of selecting at least one protein from said subpopulation comprises: screening said subpopulation against a gp120:CD4 complex; and selecting at least one protein that does not bind to said gp120:CD4 complex.

In some embodiments, the method further comprises: conjugating the protein that binds CD4 but does not bind to the gp120:CD4 complex to an adjuvant.

In some embodiments, the method further comprises: using the protein that binds CD4 but does not bind to the gp120:CD4 complex to prepare antibodies to the region of CD4 that binds gp120.

Various embodiments of a method of the present teachings comprise: screening a library comprising proteins linked to their cognate mRNAs to identify mRNA-protein pairs having substantial affinity for CD4, wherein said mRNA-protein pairs do not bind a gp120:CD4 complex; isolating one or more proteins from the identified mRNA-protein pairs; and conjugating the isolated protein(s) to an adjuvant.

In some embodiments, the method further comprises: administering the isolated protein(s) conjugated to the adjuvant to a mammal to stimulate an immune response.

In some embodiments, the method further comprises: preparing antibodies to said isolated protein(s).

These and other objects and features of the invention will become more fully apparent when the following detailed description is read in conjunction with the accompanying drawing.

### BRIEF DESCRIPTION OF THE DRAWINGS

FIG. 1 is a schematic depicting one embodiment of a protocol for preparing an HIV vaccine or antibodies.

### DETAILED DESCRIPTION

HIV infection begins when the virus binds to the CD4 on T helper cells through the CD4 binding site on the gp120 protein on HIV's viral envelope. The binding of CD4 by HIV is a highly conserved stage of the viral cycle common to all HIV strains. The affinity of HIV for the CD4 determinant is very high, on the order of 10⁻⁹ M (Nature 331:84-86, 1988). Prevention of this coupling is key to preventing the infection because, as the initiation step in the infection process, it can circumvent HIV's other defenses.

CD4 is a receptor on T cells. CD4 has a cytoplasmic tail, one transmembrane region, and four extracellular domains. All of the gp120-binding residues are located within the first N-terminal domain of CD4. Sharma et al., Biochemistry, 44 (49), 16192-16202, 2005. Most residues in CD4 that interact with gp120 lie within the region 21-64 of domain D1 of CD4. Sharma et al.

In some embodiments, methods for producing proteins that bind the CD4 determinant are provided. These proteins can be used to produce antibodies that block CD4 binding to gp120. In some embodiments, the methods include preparing a library of soluble proteins, and screening the proteins for binding to CD4. Soluble forms of CD4 are available and have been shown to inhibit HIV (Nature 334:444-447, 1988, and Nature 331:84-86, 1988). In some embodiments, the screening can be carried out on, for example, a solid substrate.

In some embodiments, the method further comprises collecting the proteins that bind CD4. In some embodiments, proteins that specifically bind the CD4 determinant, i.e., the region of CD4 that binds gp120, can be selected out of the population of CD4 binders by, for example, blocking the CD4 determinant with gp120 and then selecting the proteins that do not bind the gp120:CD4 complex. Under these conditions, any proteins that bind are discarded because they can bind antigenic determinants other than the CD4 determinant. The proteins that remain in solution are selected as binding the CD4 determinant specifically. In some embodiments, these remaining soluble proteins can be used as the antigen to make HIV vaccine. In other embodiments, these remaining soluble proteins can be used to prepare therapeutic antibodies. Such therapeutic antibodies can be used, for example, to treat or prevent HIV infection and AIDS.

In some embodiments, a population of hydrophilic proteins that bind to the CD4 determinant is provided by the methods described above. Such proteins present an epitope that is a negative impression of the CD4. A population of proteins that have this negative impression but are otherwise unrelated enables presentation of a surface antigen that having one epitope in consensus.

In some embodiments, antibodies against gp120 are provided. In some embodiments, the antibodies are produced against a population of hydrophilic proteins that bind to the CD4 determinant. The antibodies are highly similar to the CD4 viral receptor, i.e., the antibodies have an idiotype that mimics CD4 and will therefore are a match for the CD4 binding site on the gp120 on HIV. Blocking the gp120 on HIV with CD4 idiotype antibodies can prevent the critical initial step of HIV infection thus prevent infection.

### Some Definitions

As used herein, “CD4” means any CD4 protein encoded by a naturally occurring CD4 gene. CD4 was initially described as a cell surface marker for T-helper lymphocytes. CD4 was subsequently found to be expressed sparsely on monocytes, Langerhans, microglial cells, and subsets of B cells. The CD4 molecule was found also to participate directly in activation of antigen-specific T helper cells through its function as a receptor for the MHC class II molecule. In 1984, human CD4 was found to be the receptor for HIV (Dalgleish et al., Nature, 1984, 312:763). Binding of HIV envelope glycoprotein, gp120, to CD4 represents the initial step in viral entry into the target cell. The amino acid sequence for human CD4 is incorporated herein from Maddon et al. (Cell, 1985; 42:93; and, Littman et al., Cell, 1988; 55:541).

The term “CD4 determinant” refers to the region of CD4 that binds gp 120.

Immunoglobulin molecules consist of heavy (H) and light (L) chains, which comprise highly specific variable regions at their amino termini. The variable (V) regions of the H (V_{H}) and L (V_{L}) chains combine to form the unique antigen recognition or antigen combining site of the immunoglobulin (Ig) protein. The variable regions of an Ig molecule contain determinants (i.e., molecular shapes) that can be recognized as antigens or idiotypes.

The term “epitope” refers to the set of antigenic or epitopic determinants (i.e., idiotopes) of an immunoglobulin V domain (i.e., the antigen combining site formed by the association of the complementarity determining regions or V_{H} and V_{L} regions).

The term “idiotope” refers to a single idiotypic epitope located along a portion of the V region of an immunoglobulin molecule.

The term “immune effector” refers to a molecule, or derivatives, fragments, or subunits thereof, able to stimulate an immune response in the subject being treated, and may comprise an antibody, or derivatives, fragments, or subunits thereof, or a non-antibody molecule.

An “adjuvant” is a compound which enhances or stimulates the immune response when administered with one or more antigen(s).

The terms “protein,” “peptide,” and “protein” are defined herein to mean a polymeric molecule of two or more units comprised of amino acids in any form (e.g., D- or L-amino acids, synthetic or modified amino acids capable of polymerizing via peptide bonds, etc.), and these terms may be used interchangeably herein.

As used herein, the term “pharmaceutically acceptable carrier” is intended to mean a medium having sufficient purity and quality for use in humans. Such a medium can be a human pharmaceutical grade, sterile medium, such as water, sodium phosphate buffer, phosphate buffered saline, normal saline or Ringer's solution or other physiologically buffered saline, or other solvent or vehicle such as a glycol, glycerol, an oil such as olive oil or an injectable organic ester. Pharmaceutically acceptable media are substantially free from contaminating particles and organisms.

As used herein, “therapeutically- or pharmaceutically-effective amount” as applied to the disclosed compositions refers to the amount of composition sufficient to induce a desired biological result. That result can be alleviation of the signs, symptoms, or causes of a disease, or any other desired alteration of a biological system. For example, the result can involve a decrease and/or reversal of cancerous cell growth.

### Various Embodiments

Provided herein, are methods for producing proteins that bind the CD4 determinant (the region of CD4 that binds gp120). Also provided are methods for preparing HIV vaccines and anti-CD4 determinant antibodies. The vaccines are designed to stimulate an immune response to the region of gp120 that binds CD4.

In some embodiments, to produce proteins that bind the CD4 determinant, a library of proteins can be screened to identify and select proteins that bind CD4. The proteins identified as binding to CD4 can subsequently be screened against binding the gp120:CD4 complex to identify and select proteins that do not bind the gp120:CD4 complex. Proteins that bind CD4 but do not bind the gp120:CD4 complex can be used, for example, as antigen for an HIV vaccine or to product antibodies having a CD4 idiotype.

In some embodiments, a library of proteins can be a library of soluble proteins linked to their cognate mRNA, as described in, for example, PCT Application Nos. PCT/US2008/053757, filed Feb. 12, 2008, and PCT/US2006/000956, filed Jan. 12, 2006, each of which is incorporated herein by reference in its entirety.

In various embodiments, cognate pairs are selected based on one or more desired characteristics. In some embodiments, the selection of cognate pairs is based upon the binding of a CD4. In other embodiments, the selection of cognate pairs is based upon the absence of binding to the 120:CD4 complex. Binding can be determined by any of a variety of established methods, including, but not limited to, arrays, affinity columns, immunoprecipitation, and the like. In some preferred embodiments, selection criteria are measured using a high throughput screening procedure. The selection can be positive or negative in various embodiments, according to the desired characteristics of the protein.

In some preferred embodiments, cognate pairs are assayed for binding to a ligand of interest, such as CD4, or gp120:CD4 complex. In preferred embodiments, a protein of interest binds CD4 but does not bind the gp120:CD4 complex. mRNA can be isolated for proteins exhibiting the desired binding characteristics, and large quantities of the protein can be produced using standard molecular cloning techniques known in the art. The protein of interest can then be incorporated into a vaccine to target HIV.

In some preferred embodiments, the protein of interest can be used in compositions for immunizing a mammal against HIV. In some embodiments, an oligonucleotide or polynucleotide, encoding a desired antigen, can be administered with the protein antigens for in vivo expression.

In some embodiments, antibodies against the region of gp120 that binds to CD4 are provided. Antibodies useful in the invention may be derived from any mammal, or may be a chimeric antibody derived from a combination of different mammals. The mammal may be, for example, a rabbit, a mouse, a rat, a goat, or a human. The antibody is preferably a human antibody. Reactivity of antibodies against a target antigen may be established by a number of well known means, including Western blot, immunoprecipitation, ELISA, and FACS analyses using, as appropriate, Fab idiotype fragments, peptides, idiotype-expressing cells or extracts thereof. The antibody can belong to any antibody class and/or sub-class. The antibodies may also contain fragments from antibodies of different classes and sub-classes, thereby forming a composite.

In some embodiments, human monoclonal antibodies that have a CD4 idiotype (i.e., antibodies that bind to the region of gp120 that binds to CD4) are produced using methods known in the art (for review, see Vaughan et al., 1998, Nature Biotechnology 16: 535-539), for example by screening a phage display library, as described, e.g., in Parmley and Smith Gene 73:305-318 (1988), Barbas et al., Proc. Natl. Acad. Sci. USA 88: 7978-7982 (1991), Griffiths et al., EMBO J 13: 3245-3260 (1994), Griffiths and Hoogenboom, Building an in vitro immune system: human antibodies from phage display libraries. In: Protein Engineering of Antibody Molecules for Prophylactic and Therapeutic Applications in Man. Clark, M. (Ed.), Nottingham Academic, pp 45-64 (1993), and Burton and Barbas, Human Antibodies from combinatorial libraries. Id., pp 65-82, all of which are herein incorporated by reference. Typically, clones corresponding to antibodies which produce binding affinities of a desired magnitude are identified, and the DNA is used to produce the antibodies of interest using standard recombinant expression methods.

Fully human monoclonal antibodies may also be produced using transgenic mice engineered to contain human immunoglobulin gene loci, as described in PCT Patent Application WO98/24893 and Jakobovits, 1998, Exp. Opin. Invest. Drugs 7(4): 607-614, herein incorporated by reference. This method avoids the in vitro manipulation required with phage display technology and efficiently produces high affinity authentic human antibodies.

In some embodiments, an antibody against an antigen of interest, such as a protein that binds the CD4 determinant, is produced in the patient selected for treatment with the modular therapeutic. For example, in some embodiments, a subject is “vaccinated” with the antigen of interest, and antibodies from the subject against the antigen are selected by binding to the antigen of interest, as described in Zebedee, et al. Proc. Natl. Acad. Sci. USA 89: 3175-3179 (1992), Burton et al., Proc. Natl. Acad. Sci. USA 88: 10134-10137 (1991), and Barbas et al., Proc. Natl. Acad. Sci. USA 89: 10164-20168 (1991).

In some embodiments, additional rounds of screening are performed to increase the affinity of the originally isolated antibody. For example, in some embodiments, the affinity of the antibody is enhanced by affinity maturation, in which hypervariable antibody regions are mutated to produce a large number of combinations, and the corresponding antibody variants are screened via phage display to select antibodies having the desired affinity for the antigen. In further embodiments, the small protein epitope can undergo in vitro evolution, as described in more detail below, to increase its binding affinity for the antibody. Advantageously, in some embodiments, for example those in which antibodies are produced by “vaccinating” a subject, an analogous process is carried out by the host immune system (e.g., via clonal selection) to produce high affinity antibodies specific for the antigen of interest.

Some embodiments of the invention also provide pharmaceutical compositions containing an antigen or antibody and a pharmaceutical carrier. Such compositions can be used, for example, to prevent, treat or reduce the severity of, or ameliorate a sign and/or symptom associated with, HIV infection. For example, proteins that bind the CD4 determinant can be administered as a solution or suspension together with a pharmaceutically acceptable medium.

Formulations containing an antigen or antibody of the invention include those applicable for parenteral administration such as subcutaneous, intraperitoneal, intramuscular, intravenous, intradermal, intracranial, intratracheal, and epidural administration. Additional formulations are applicable for oral, rectal, ophthalmic (including intravitreal or intracameral), nasal, topical (including buccal and sublingual), intrauterine, or vaginal administration. A formulation containing an antigen or antibody can be presented in unit dosage form and can be prepared by pharmaceutical techniques well known to those skilled in the art. Such techniques include the step of bringing into association the active ingredient and a pharmaceutical carrier or excipient.

In some embodiments, a protein that binds the CD4 determinant can be configured in a liposome format. For example, the proteins can be presented on the surface of liposomes. The use of liposomes allows creation of a two compartment vaccine. For example, the surface of the liposome includes proteins with the negative impression of the CD4, and inside the liposome are proteins that will be liberated on endocytosis and cleaved to give the MHC II ligand that will be displayed on the surface of the antigen presenting cells to activate T helper cells.

In some embodiments, a portion of the liposomes adhere to the T cells, and a portion of the liposomes are taken up by antigen presenting cells. With liposomes, drugs can be encapsulated within a relatively impermeable bilayer membrane where the drug is protected from the environment. Liposomes can be taken up by cells, and therefore, because of the close proximity, the cellular uptake of the encapsulated drug is enhanced. Liposomes are natural and biodegradable (M. J. Ostro, ed., Marcel Deckker, Inc., pp 289-341, 1983). Liposomes have been used successfully to study HIV infections (Biochim. Biophys. Res. Comm. 159:566-571, 1989) or to demonstrate that reconstituted CD4 in liposomes reacted with HIV-infected cells in vitro (J. Acquired Immune Deficiency-Syndromes 3:109-114, 1990).

In some embodiments, the liposomes can contain lipid A adjuvant. Immunogenicity of protein and peptide antigens encapsulated in liposomes was demonstrated to be significantly enhanced by including lipid A with the antigens (Abstract, Liposomal subunit vaccines: Effects of lipid A and aluminum hydroxide on immunogenicity. Conference on Formulations and Drug Delivery, held on Oct. 10-13, 1995, in Boston, Mass.).

For preventing, treating or reducing the severity of HIV infection, an effective amount of an HIV vaccine or HIV antibody is administered to a subject. An effective amount is an amount capable of reducing the risk of HIV infection or an amount such that an effect of HIV infection pathologically reduced. An effective amount can be, for example, between about 10 μg/kg to 500 mg/kg body weight, for example, between about 0.1 mg/kg to 100 mg/kg, or preferably between about 1 mg/kg to 50 mg/kg, depending on the treatment regimen. For example, if a agent or formulation containing the agent is administered from one to several times a day, then a lower dose would be needed than if a formulation were administered weekly, or monthly or less frequently. Similarly, formulations that allow for timed-release of the agent, such as those described herein, would provide for the continuous release of a smaller amount of the agent than would be administered as a single bolus dose. For example, a agent of the invention can be administered at between about 1-5 mg/kg/week.

In some embodiments, administration of a formulation of an antigen or antibody can be, for example, simultaneous with or delivered in alternative administrations with the conventional HIV therapy, including multiple administrations. Simultaneous administration can be, for example, together in the same formulation or in different formulations delivered at about the same time or immediately in sequence. Alternating administrations can be, for example, delivering an antigen or antibody and a conventional HIV therapeutic treatment in temporally separate administrations. As described herein, the temporally separate administrations of an agent (e.g., a vaccine or antibody) and conventional therapy can similarly use different modes of delivery and routes.

The following Examples illustrate various embodiments of the present invention and are not intended in any way to limit the invention.

### Example 1

### Preparation of Antigen for HIV Vaccine

This example illustrates one possible method for producing proteins that bind the CD4 determinant are provided. These proteins are used to produce antibodies that block CD4 binding to gp120.

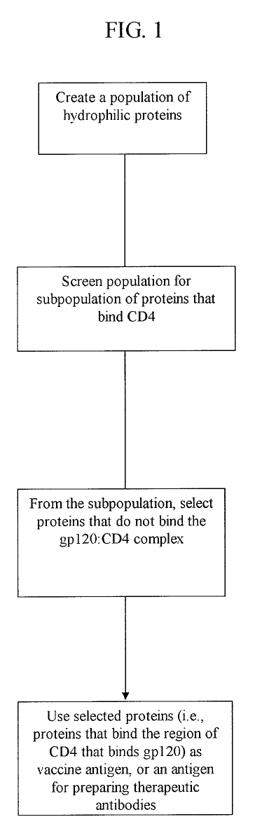

A library of soluble proteins where each protein is linked to its cognate mRNA is prepared (FIG. 1 at 10). The library of soluble proteins is screened to identify proteins that bind CD4 (FIG. 1 at 20). Proteins that bind CD4 are then screened to identify and select proteins that do not bind the gp120:CD4 complex (FIG. 1 at 30). Thus, the selected proteins bind to the region of CD4 that binds to gp120. The cognate mRNA of the selected proteins is used to make protein using host organisms and standard bioreactor culture conditions. The prepared protein is then used as antigen for an HIV vaccine (FIG. 1 at 40).

### Example 2

### Liposome HIV Vaccine Formulations

This example illustrates the HIV vaccine in liposome format.

For the vaccine antigen, proteins that bind the CD4 determinant are produced as described herein. Liposomes composed of DMPC:DMPG:CHOL:LA (9:1:7.5:0.11) are added to the emulsified o/w composition which contained no other stabilizer. The liposomal formulation will contain, for example without limitation, about 10-1000 μg of antigen per, for example without limitation, about 0.2-3 ml of liposome suspension. Approximately 10-1000 μg PSA with about 20-1000 μg lipid A is formulated in 0.1-5 ml of liposomes. For example, liposomes can be prepared using DMPC:DMPG:cholesterol at molar ratios of, for example, 9:1:7.5 and lipid A can be added to obtain a final concentration of, for example, 200 μg/ml. The lipid mixture is added to water and vortexed and then lyophilized. To the lyophilized preparation, sufficient antigen is added in buffer (20 mM tris-glycine, 150 mM NaCl and 0.02% Tween 80 pH 7.4) to provide the desired concentration. Antigen at a concentration of about 0.1-1 μg/ml was added to the lipophilized liposomes so that the final phospholipid concentration was about 200 mM phosphate. The mixture is vortexed to encapsulate the antigen and the liposomes were washed and again suspended.

The suspension is used directly as a vaccine or used to prepare the emulsion.

For preparation of the emulsion, about 0.1-10 ml of the liposome suspension is thoroughly mixed with about 0.05-1 ml mineral oil using the syringe extrusion method. Briefly, the mineral oil and suspension are placed in separate syringes which were connected via tubing. The liquids were pulsed back and forth until an adequate suspension was obtained. The suspension is immediately used as a vaccine.

With the liposome vaccines, antigen will be presented on the surface of liposomes as well as within the liposomes. Liposome surface proteins provide binding sites to CD4, thereby blocking binding of HIV to the CD4 on T cells. Proteins within liposomes will be liberated on endocytosis and cleaved to give the MHC H ligand that will be displayed on the surface of the antigen presenting cells to activate T helper cells. Thus, some liposomes will adhere to the T cells, and other liposomes will be taken up by antigen presenting cells.

### Example 3

### Testing of the HIV Vaccine

Initial antibody responses to the HIV vaccine prepared by the methods disclosed herein will be determined using BALB/c mice inoculated intramuscularly with the vaccine. The vaccine will then be tested in humanized NOD/SCID/IL2Rγnull mice transplanted with human hematopoietic stem cells (J. of Virology, 81(23):13259-13264, 2007). This is the most advanced mouse model available for studying HIV infection. The mice will be immunized with the vaccine and then will be challenged with wild type HIV-1 and with HIV-1 subtypes A, B, and C obtained from the NIH AIDS Research & Reference Program. Levels of CD4+ and CD8+ T cells in the challenged mice as compared to negative and positive controls will be monitored with high resolution flow cytometry to determine the vaccine's effectiveness.

Subsequent further development and testing of the vaccine will be carried out in simian models.

### INCORPORATION BY REFERENCE

All references cited herein, including patents, patent applications, papers, text books, and the like, and the references cited therein, to the extent that they are not already, are hereby incorporated by reference in their entirety. In the event that one or more of the incorporated literature and similar materials differs from or contradicts this application; including but not limited to defined terms, term usage, described techniques, or the like, this application controls.

### EQUIVALENTS

The foregoing description and Examples detail certain preferred embodiments of the invention and describes the best mode contemplated by the inventors. It will be appreciated, however, that no matter how detailed the foregoing may appear in text, the invention may be practiced in many ways and the invention should be construed in accordance with the appended claims and any equivalents thereof.

1. A method for making an antigenic composition that raises antibodies against HIV, the method comprising:
preparing a population of soluble proteins;
reacting a CD4 protein with a gp120 protein to generate a blocked CD4 protein comprising a gp120:CD4 complex;
screening said population of soluble proteins for binding to said blocked CD4 protein;
identifying a subpopulation of proteins from said population, wherein said subpopulation of proteins comprises proteins that do not bind to said blocked CD4 protein; and
selecting at least one protein from said subpopulation;
screening said at least one protein from said subpopulation for binding to an unblocked CD4 protein
identifying at least one protein that binds to said unblocked CD4 protein,
wherein said at least one protein that binds to said unblocked CD4 protein is used as an antigen for generating said antigenic composition.

2. The method of claim 1, wherein said population of soluble proteins comprises proteins linked to their cognate mRNA.

3. The method of claim 1, wherein said proteins linked to their cognate mRNA are about 100 amino acids in length.

4. The method of claim 1, wherein said blocked CD4 protein is attached to a solid substrate.

5. (canceled)

6. The method of claim 1, further comprising conjugating said at least one protein from said subpopulation to an adjuvant.

7. The method of claim 1, further comprising using at least said at least one protein to prepare antibodies to the region of CD4 that binds gp120.

8. A method for making a therapeutic for preventing or treating HIV, the method comprising:
screening a library comprising proteins linked to their cognate mRNAs to identify mRNA-protein pairs having substantial affinity for CD4, wherein said mRNA-protein pairs do not bind a gp120:CD4 complex;
isolating one or more proteins from the identified mRNA-protein pairs; and
conjugating the isolated protein(s) to an adjuvant.

9. The method of claim 8, further comprising administering said isolated protein(s) conjugated to the adjuvant to a mammal to stimulate an immune response.

10. The method of claim 8, further comprising preparing antibodies to said isolated protein(s).

11. A method for preventing or treating HIV in a subject comprising:
preparing an HIV vaccine according to the method of claim 1; and
administering said vaccine to a subject;
wherein said vaccine induces production of antibodies in said subject that recognize the gp120 protein of HIV, and
wherein binding of HIV by said antibodies results in HIV neutralization, thereby preventing or treating HIV.

12. A method for making an antigenic composition that raises antibodies against HIV, the method comprising:
preparing a population of soluble proteins;
screening said population of soluble proteins for binding to an unblocked CD4 protein;
identifying a subpopulation of proteins from said population, wherein said subpopulation of proteins comprises proteins that bind to said unblocked CD4 protein;
reacting a CD4 protein with a gp120 protein to generate a blocked CD4 protein comprising a gp120:CD4 complex;
screening said subpopulation of proteins that bind to said unblocked CD4 protein for binding to said blocked CD4 protein;
identifying a second subpopulation of proteins from said subpopulation, wherein said second subpopulation of proteins comprises proteins that do not bind to said blocked CD4 protein; and
selecting at least one protein from said second subpopulation;
screening said at least one protein from said second subpopulation for binding to a second unblocked CD4 protein identifying at least one protein from said second subpopulation that binds to said second unblocked CD4 protein,
wherein said at least one protein that binds to said second unblocked CD4 is used as an antigen for generating said antigenic composition.
